(19) United States
(12) Patent Application Publication
(10) Pub. No.: US 2010/0331727 A1
(43) Pub. Date: Dec. 30, 2010

(54) Linear Device for Microdialysis, Method of Manufacturing the Device and Method for Studying a Tissue with the Device

(76) Inventors: Stefan Lindgren, Vallentuna (SE); Anton Karlsson, Enskede (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/667,801

(22) PCT Filed: Jul. 9, 2008
(86) PCT No.: PCT/SE08/50854

§ 371 Date: Jan. 5, 2010

Related U.S. Application Data
(60) Provisional application No. 60/958,935, filed on Jul. 10, 2007.

(30) Foreign Application Priority Data
Jul. 10, 2007 (SE) 0701713-0

(51) Int. Cl. A61B 5/00; A61M 31/00; B21D 51/16

(52) U.S. Cl. 600/582; 604/500; 29/890.09

(57) ABSTRACT

There is provided a linear device (100) for microdialysis, comprising a length of tubing (102) having at least one lumen (118) wherein at least one tubular membrane sleeve (104) is coaxially arranged outside the tubing periphery and extending at least partially along the periphery of the tubing (102). The membrane sleeve (104) is extending intermediate the ends of the tubing (102), and the tubing (102) extends at least through the tubular membrane sleeve (104). There is also provided a method of manufacturing the linear device (100) and a method wherein the linear device (100) is used.

### TECHNICAL FIELD

The present invention relates to the field of linear devices, more specifically to a linear device for microdialysis. It also relates to a method for manufacturing a linear device for microdialysis and to the use of a linear device for microdialysis.

### BACKGROUND

Microdialysis is used to monitor interstitial fluid in various body organs with respect to local metabolic changes. Linear devices for microdialysis, e.g. catheters or probes, may for example be used to monitor interstitial fluid in body tissue, e.g. the skin or muscles. When a linear catheter or probe is placed in e.g. skin tissue it is subjected to substantial strain, both when introducing (depending on the method used for introducing) and removing or pulling out the catheter or probe. Since the semi-permeable membrane used in catheters or probes for microdialysis is very fragile, there is s risk that the membrane will be damaged during removal and/or insertion of the catheter or probe for microdialysis.

In a probe for microdialysis a perfusion liquid flows on the inner side of a semi-permeable membrane and molecules from the tissue surrounding the membrane enters through the membrane through diffusion. After the molecules have entered the liquid it is often called dialysate.

In U.S. Pat. No. 5,706,806 it is shown a linear microdialysis probe having a support fibre 12. This design is not entirely satisfactory since preferably the support fibre 12 is pulled out after insertion of the probe. If the support fibre 12 would be left in the probe during use it would reduce the flow area in the probe. When the probe is pulled out after use, the membrane will then be subjected to the full pulling strain which may damage the membrane. Moreover, the use of the support fibre 12 makes the probe rather complex.

The document US 2003/0 236 454 relates to a non-linear microdialysis probe with a plurality of lumen and a rather large diameter of about 2 mm. Because of the large diameter this probe is not possible to use in human skin tissue which is only about 1 mm in thickness.

It is an aim to provide a linear device that obviates or at least reduces some or all of the drawbacks connected with the background art.

### SUMMARY

With a linear device for microdialysis is meant a device where the fluid flow substantially occurs in one main direction through the device. That is, the fluid enters a lumen of the linear device at a proximal end, substantially follows a first main flow direction through the device and also substantially follows the first main flow direction when exiting the device at a distal end of the device. However, on its way from the proximal end towards the distal end, the fluid may for shorter distances flow in a direction different from the main direction.

In contrast, in a conventional device or catheter for microdialysis, the fluid enters a lumen of the device at a proximal end and flows in a first direction. The fluid then reaches the distal end where the fluid flow changes direction to a second direction substantially opposite to the first direction and flows back in a second lumen towards the proximal end.

In this application the word lumen is used in the meaning of a channel or space adapted so that a fluid can flow there through or therein.

Generally a linear device for microdialysis may comprise a length of tubing having at least one lumen. At least one tubular, sleeve-formed membrane is coaxially arranged outside the tubing periphery and extending at least partially along the periphery of the tubing. The membrane is extending intermediate the ends of the tubing and the tubing extends at least through the membrane.

Generally, there may also be provided a linear device for microdialysis, comprising at least one dialysis chamber, a length of tubing having at least one lumen, at least one first element and at least one second element connecting one of said at least one lumen with one of said at least one dialysis chamber, wherein at least one tubular, sleeve-formed membrane is coaxially arranged outside the tubing periphery and extending at least partially along the periphery of the tubing, intermediate the ends of the tubing, and wherein the tubing extends at least through the membrane.
The described linear device may optionally have the following further characteristics.

In one aspect a linear device for microdialysis is provided wherein the tubing imparts sufficient rigidity to the linear device in order to eliminate the need for using supporting wires to support or protect the membrane when locating the linear device in target tissue.

In one aspect a linear device for microdialysis is provided wherein the membrane extends sufficiently long along the periphery of the tubing to establish a suitable interaction between a perfusion fluid transported in the lumen and a surrounding body fluid. The membrane extends sufficiently long along the periphery of the tubing so that sufficient material can be collected from a target tissue and transported with the perfusion fluid for subsequent analysis.

In another aspect a linear device for microdialysis is provided wherein the membrane is arranged on the tubing to provide a dialysis chamber between the outer surface of the length of tubing and the inner surface of the membrane.

In a further aspect a linear device for microdialysis is provided wherein the at least one first element comprises at least one inlet channel and the at least one second element comprises at least one outlet channel connecting one of the at least one lumen with one of the at least one dialysis chamber.

In one aspect a linear device for microdialysis is provided wherein there is provided at least one blocking in the tubing. The blocking is advantageously located between the at least one first element or inlet channel and the at least one second element or outlet channel. Said at least one blocking is blocking the lumen for passage of liquid.

There may also be provided more than one blocking, the blockings being located between the at least one first element or inlet channel and the at least one second element or outlet channel.

Generally, a method of manufacturing a linear device for microdialysis may comprise the following steps:
a) providing a length of tubing having at least one lumen,
b) providing at least one first element or inlet channel extending from the outside surface of the length of tubing to the at least one lumen,
c) providing at least one second element or outlet channel at a distance from the at least one first element or inlet channel, the at least one second element or outlet channel extending from the outside surface of the length of tubing to the at least one lumen,
d) providing at least one blocking between said at least one first element or inlet channel and said at least one second element or outlet channel, said blocking stopping the passage of liquid in the lumen,
e) arranging a tubular, sleeve-formed membrane coaxially outside the tubing periphery and extending at least partially along the periphery of the tubing, said membrane covering the at least one first element or inlet channel and the at least one second element or outlet channel,
f) sealingly attaching the membrane to the tubing.
The method of manufacturing a described linear device may optionally have the following further characteristics.
   In another aspect there is provided a method of manufacturing a linear device for microdialysis wherein the membrane is sealingly attached to the tubing by means of attachment means such as e.g. adhesive material, e.g. glue or polymer.

In yet another aspect, a method for studying a tissue, for example for studying the local effect of a drug substance in a tissue and/or for studying the condition in a tissue, is provided. Said method may comprise the following steps:
a) invasively locating a linear device as described herein, in a target tissue,
b) providing a perfusion fluid in a lumen of said linear device,
c) permitting interaction between the perfusion fluid and the tissue by a membrane of said linear device,
collecting perfusion fluid having interacted with the tissue for analysis.

The method for studying a tissue may optionally have the following further characteristics.

In another aspect there is provided a method for studying a tissue wherein the linear device is used to study skin tissue.

In a further aspect there is provided a method for studying a tissue wherein the linear device is used to administer substances to a tissue where the effect of the substance is studied by means of the linear device.

In yet another aspect there is provided a method for studying a tissue wherein a first linear device is used to administer a drug substance to the target tissue and a second linear device is used to study the effect of the drug substance.
Further possible features and benefits of the present linear device and methods described herein will be explained in the detailed description below.

### BRIEF DESCRIPTION OF THE DRAWINGS

The invention will now be described by way of non limiting embodiments with reference to the accompanying drawings in which:

FIGS. 1a and 1b are a plan view respectively a cross section of a linear device as described herein, according to a first embodiment, in section H—H,

FIGS. 1c and 1d are a plan view respectively a cross section of a linear device as described herein, according to a first embodiment, in section J-J,

FIGS. 2a, 2b are a plan view and a cross section of a linear device as described herein, according to a second embodiment comprising two blockings 110,

FIGS. 3a and 3b are a plan view respectively a cross section of a linear device as described herein, according to a third embodiment comprising four outlet channels, in section H—H,

FIGS. 3c and 3d are a plan view respectively a cross section of a linear device as described herein, according to a third embodiment comprising four outlet channels, in section J-J,

FIGS. 4a and 4b are a plan view respectively a cross section of a linear device as described herein, according to a fourth embodiment comprising two oval outlet channels, in section H—H,

FIGS. 4c and 4d are a plan view respectively a cross section of a linear device as described herein, according to a fourth embodiment comprising two oval outlet channels, in section J-J,

FIG. 5 is a drawing showing a second embodiment of the blocking of the lumen 118 in the tube 102,

FIG. 6 is a drawing schematically showing the linear device 100 introduced into tissue, e.g. skin tissue.

FIGS. 7a and 7b schematically shows a linear device 200 with two lumen 118 and 132.

### DETAILED DESCRIPTION

Before the device, method and use described herein is described in detail, it is to be understood that this device, method and use is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” “an” and “the” also include plural referents unless the context clearly dictates otherwise. Thus, for example, reference to “an element” includes more than one such element, and the like.

In this specification and in the claims which follow, reference will be made to a number of terms which shall be defined to have the following meanings:

The linear device 100 described herein enter the body at one point, thread through the tissue of interest, and then exit the body at a second point. The linear design has the advantage of minimizing tissue damage, because of its small diameter or height, being very flexible and therefore more comfortable, and durable due to the construction as disclosed herein.

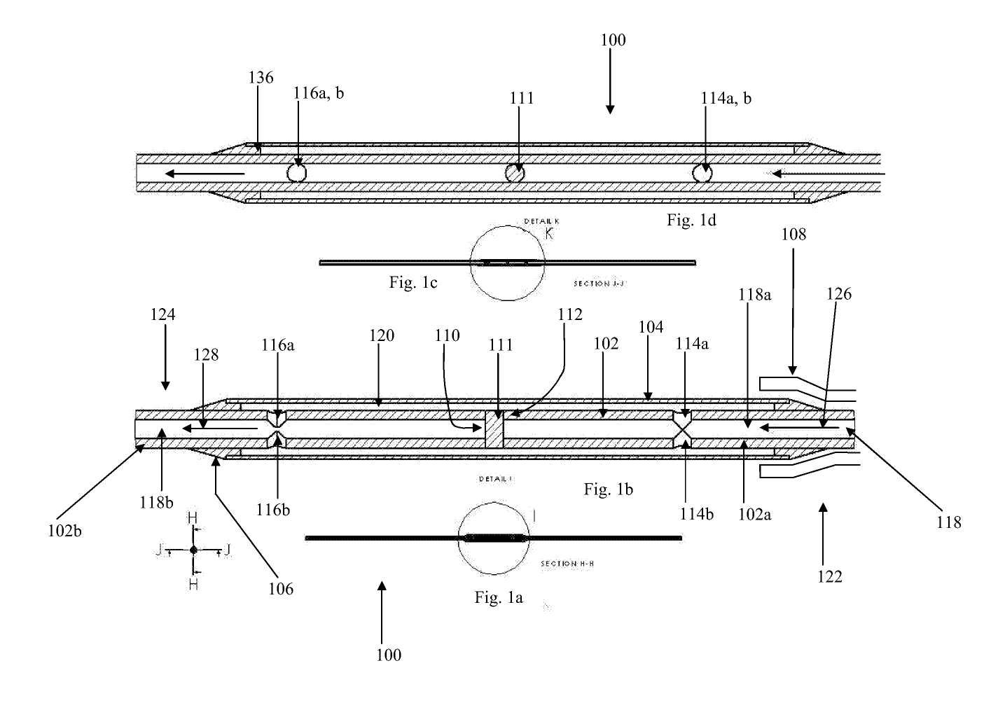

Relating to FIGS. 1-5 different embodiments of a linear device 100 as described herein will now be described. For the sake of clarity are not all reference signs present in all figures. The linear device 100 may advantageously be used for introduction in skin tissue, but also in muscle tissue, or other applications, especially applications where the advantages of the described linear device 100 is an advantage. One advantage of the linear device 100 is e.g. the small outer diameter, or height if the linear device would have a oval section.

One example of an advantageous application is clinical studies where the effect of substances, e.g. drugs, may be tested or measured locally in skin tissue or other tissues of interest. With the linear device 100 substances, e.g. drugs, may also be administrated to the body, e.g. to skin tissue or other tissues of interest. The administration of a substance takes place through diffusion, the substance travels from the perfusion fluid 126 to the body through diffusion through the membrane 104. A linear device 100 for microdialysis as described herein is sometimes called linear microdialysis probe or linear microdialysis catheter.

As seen in FIGS. 1-5 the device 100 comprises a tube 102 and a hollow or sleeve-formed, tube-shaped semi-permeable membrane 104 where the tube 102 extends through the membrane 104 in the longitudinal direction of the membrane 104. The membrane 104 is coaxially arranged outside the tube or tubing periphery and extending at least partially along the periphery of the tube 102.

Since the tube 102 extends through the membrane 104 the membrane 104 is supported by the tube 102. The tube 102 has characteristics, e.g. rigidity, enabling the tube 102 to support the membrane 104 so that the membrane 104 is not damaged during handling. The membrane 104 is attached or fixed to the tube 102 by means of attachment means 106, e.g. an adhesive material. Shrinking tubing 108 may be applied to the linear device 100 to make the linear device 100 less sensitive to kinking or buckling. A shrinking tube is a tube that shrinks radially when subjected to heat. Advantageously two pieces of shrinking tubing 108a and 108b are applied, each piece extending from a respective end of the tube 102 towards the membrane 104 and extending a short distance past the joint between the membrane 104 and the tube 102 so that the shrinking tubing 108 overlaps the membrane 104 with a short distance, advantageously about 0.5-1 mm. To use a shrinking tubing 108 in this way may be advantageous e.g. when the tube 102 has a small wall thickness. The wall thickness of the shrinking tubing 108 may advantageously be about one tenth of a millimetre. The shrinking tubing 108 may also be applied only to the joint between the tube 102 and the membrane 104 as a complement to the attachment means 106 to further strengthen the joint. The shrinking tubing 108 may in this case advantageously have a length of about 2 mm and extend about 1 mm past the joint on each side of the joint. The tube 102 comprises a lumen 118 comprising an inlet lumen 118a and an outlet lumen 118b. Further, the tube 102 comprises at least one inlet channel 114a, 114b and at least one outlet channel 116a, 116b and at least one plug through hole 112. The inlet and outlet channels 114a, 114b, 116a, 116b are realized as through holes 114 and 116 and connect the respective lumen 118a and 118b with a dialysis chamber 120 between membrane 104 and tube 102. The through hole 112 comprises a plug 111.

The tube 102 may have an outer diameter, or height, in the range of about 0.6-0.1 mm, advantageously about 0.38-0.18 mm, the diameter of the lumen 118 may respectively be in the range of about 400-60 micrometer and about 260-90 micrometer.

The tube 102 may e.g. be made of Polyuretan (PUR) or Polyimid (PI), both materials are available in bio compatible grades and suitable for this type of application. Both materials may be used together with an adhesive material. Using tubes of PI, tubes with lower wall thickness is possible than with PUR, PI also has lower affinity for certain drug substances than PUR has.

For PUR the lumen may have a diameter of 150 micrometer when the outer diameter of the tube is 0.38 mm. For PI the lumen may have a diameter of 125 micrometer when the outer diameter of the tube is 0.18 mm. This merely to give examples of possible dimensions.

In the direction of flow (indicated by the arrow associated with the reference numeral 126) of the perfusion fluid 126, the first part of the tube 102 may be called inlet tube 102a and the second part of the tube 102, after the membrane 104, may be called outlet tube 102b. The inlet tube 102a may have a length of about 100 mm-700 mm, advantageously about 200-500 mm, even more advantageously about 350-450 mm. The outlet tube 102b may have a length of about 20 mm-300 mm, advantageously about 30-200 mm, even more advantageously about 50-100 mm.

The length of the inlet tube 102a and outlet tube 102b may be adapted to the application in which the linear device 100 is to be used.

The membrane 104 comprises a semi-permeable hollow fibre in the form of a sleeve-formed, tubular membrane. The material for the membrane 104 may e.g. comprise polyarylethersulphone (PAES), polyethersulphone (PES) or polyamid (PA), but also other materials with suitable characteristics are possible to use. These three materials have substantially equivalent characteristics. Depending on the application for the linear device, membranes with different Molecular Weight CutOff (MWCO) are chosen. The MWCO is often measured in kiloDalton (kDa). A suitable interval for the MWCO is 5-300 kDa, this is suitable for many applications. However, the MWCO is freely chosen depending on the molecular weight of the molecules that should be analyzed with the linear device. There is a certain degree of correlation between molecular weight and size for a molecule. The membrane has a porous structure and the openings in the membrane are not well-defined channels but rather openings in the membrane that wary in size as one moves through the membrane. How large a molecule can be and still be able to pass through a membrane with a certain MWCO also depend on the shape of the molecule, and not only on the weight. The measure MWCO is therefore not absolute, it indicates that a molecule with a certain weight has a certain probability to be able to diffuse through a membrane with a specified MWCO. The length of the membrane 104 may be in the interval of 3-60 mm, advantageously 10-30 mm. The mentioned length is the active length of the membrane 104, that is, the length from the centre of the at least one inlet channel 114 to the centre of the at least one outlet channel 116. In case there are several inlet and/or outlet channels the active membrane length is the shortest distance the perfusion fluid 126 flows, i.e. the distance from the centre of the last (in the flow direction) inlet channel to the centre of the first (in the flow direction) outlet channel 116. The physical length of the membrane 104 is advantageous slightly larger than the active length. Advantageously the physical length is about 2 mm larger than the active length. The diffusion through the membrane 104 may not only be used to collect molecules from the tissue surrounding the membrane 104. The principle of diffusion may also be used to administer substances, e.g. drugs, to the tissue surrounding the membrane 104.

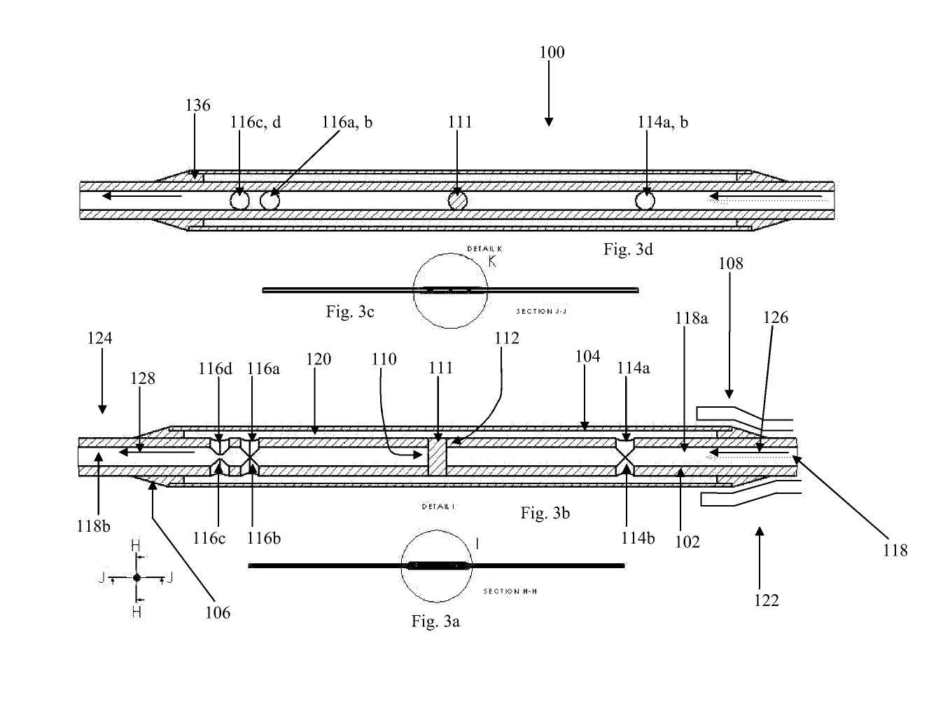

For attaching the membrane 104 to the tube 102 attachment means 106 may be used, e.g. an adhesive material such as glue or a polymer. Advantageously a bio compatible adhesive material that may be solidified with ultra violet radiation (UV-radiation) is used. To use UV-radiation to solidify the adhesive material makes it possible to solidify the adhesive material in a relatively short period of time. Advantageously the viscosity of the adhesive material is relatively high so that the adhesive material does not move too far into the dialysis chamber 120. However, the adhesive material 106 will move a short distance into the dialysis chamber 120 so as to form a thin layer of adhesive material 106 between the outer surface of the tube 102 and the inner surface of the membrane 104 (as shown at 136 in FIGS. 1d, 3d). This is advantageous since it makes the joint between the tube 102 and membrane 104 stronger. A non-limiting example of a suitable adhesive material is UV solidifying glue from Dymax Europe GmbH, Frankfurt am Main, Germany. The chosen adhesive material has an adhesion to the tube material and the membrane material that is sufficient to ensure that the membrane 104 stays attached to the tube 102, e.g. when the linear device 100 is inserted into or pulled through tissue, e.g. skin tissue.

The shrinking tubing 108 may advantageously have a wall thickness of about one tenth of a millimeter. That is, the wall thickness of the shrinking tubing 108 after the shrinking tubing 108 has been mounted, after the shrinking of the shrinking tubing 108. A non-limiting example of a suitable shrinking tubing available in bio compatible grades is polyester heat shrinking tubing from Advanced Polymers, Salem, New Hampshire, USA. Advantageously the material of the shrinking tubing 108 is chosen so that the shrinking tubing 108 may be made to shrink at a temperature that is low enough to make sure that other parts of the linear device 100 are not damaged.

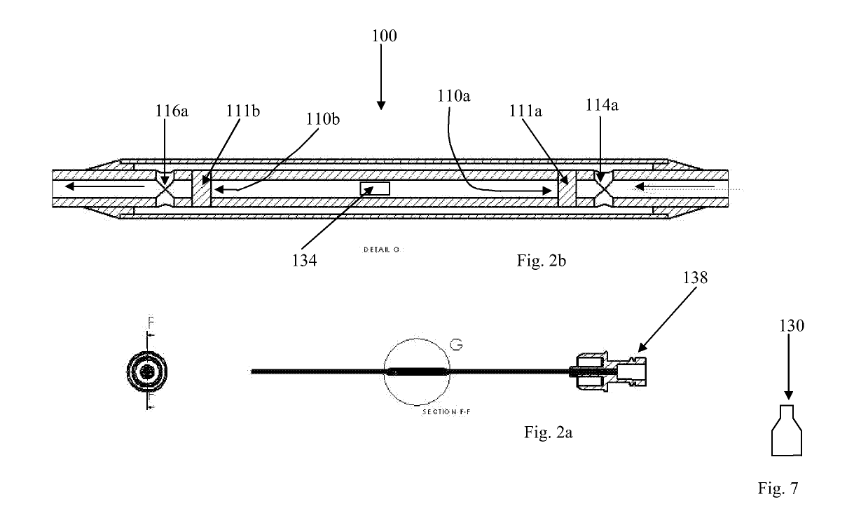

Between the at least one inlet channel 114 and the at least one output channel 116 there is provided a blocking 110 to block the passage in the lumen 118, dividing the lumen 118 into an inlet lumen 118a and an outlet lumen 118b, so that the perfusion fluid 126 must flow through the inlet channel 114 into the dialysis chamber 120. The blocking may be realized in the form of a plug 111 by feeding an adhesive material into the plug through hole 112 and then solidifying the adhesive material, e.g. by using UV-radiation or cooling an adhesive material that has been brought into a liquid state by heating the adhesive material. The adhesive material used for the plug 111 advantageously has a relatively low viscosity so that the adhesive material fills whole of the plug through hole 112. The adhesive material may fill the plug through hole 112 under the influence of capillary forces. The plug 111 has the function of blocking the inlet lumen 118a so that the perfusion fluid 126 is directed from inlet lumen 118a into the at least one inlet channel 114a, 114b. When the perfusion fluid 126 has entered the dialysis chamber 120 molecules enter the perfusion fluid 126 and the perfusion fluid 126 is called dialysate 128 as it leaves the dialysis chamber 120 and enters the output lumen 118b. When the dialysate 128 enters the output lumen 118b of course the plug 111 also forces the dialysate 128 to flow forward towards the outlet end 124. As indicated in FIG. 2b it is also possible to have two plugs 111a and 111b, one plug being provided in the vicinity of a inlet channel an the other in the vicinity of an outlet channel. This reduces the amount of stationary or non-flowing fluid in the tube 102 and shortens the response time of the linear device 100.

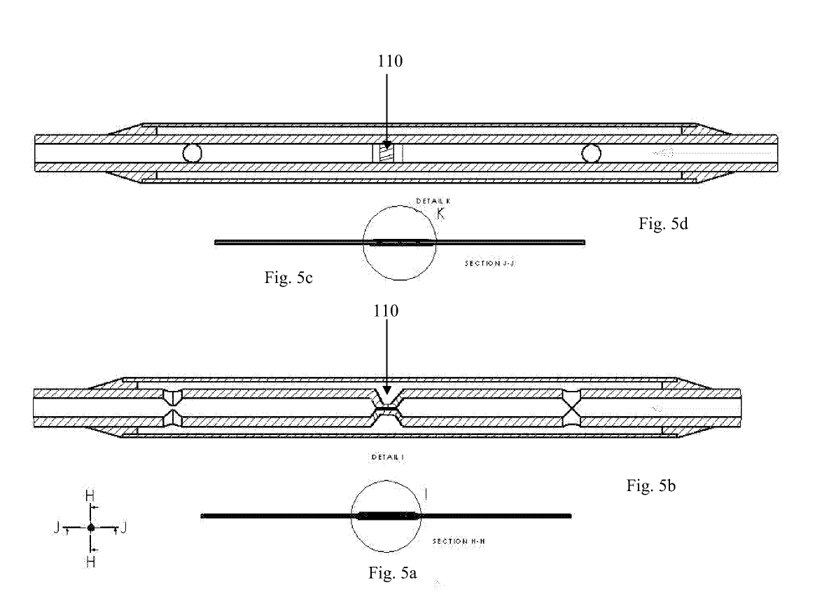

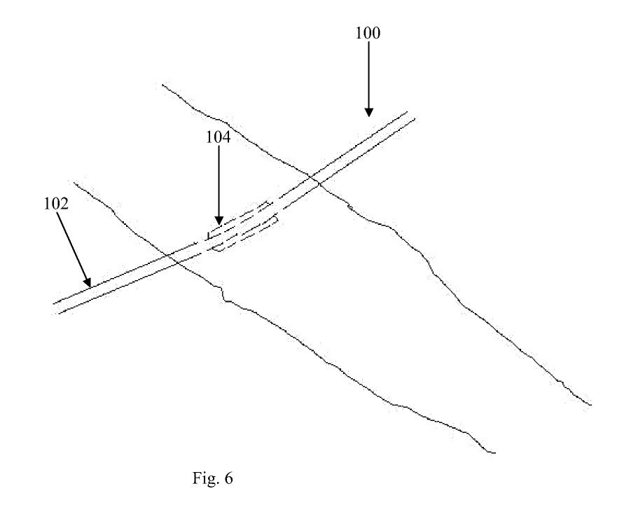

The at lest one blocking 110 between the inlet and outlet channels 114a, 114b, 116a, 116b may also be realised by applying pressure to the tube 102 so that the lumen 118 shrinks to zero and then apply heat so as to weld the inner wall of the tube 102 together. This is illustrated in FIG. 5.

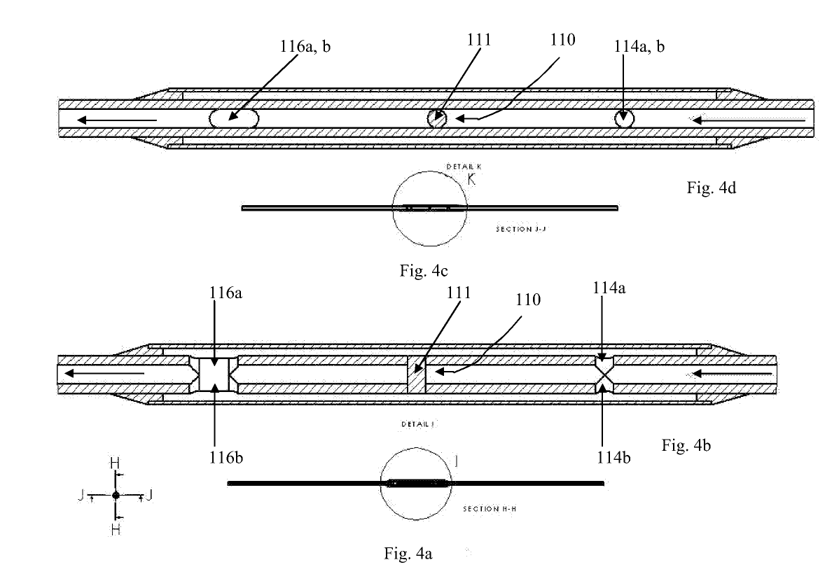

In FIG. 1b there are shown two inlet channels 114a and 114b respectively two outlet channels 116a and 116b. This is advantageous since the perfusion fluid quicker gets evenly distributed in the dialysis chamber 120 than if only one inlet channel 114a respectively one outlet channel 116a were provided. It is also possible to provide several inlet respectively outlet channels spaced circumferentially around the circumference of the tube 102, e.g. inlet channels 114a-114d and outlet channels 116a-116d. But it is also possible to use only one inlet channel 114a and one outlet channel 116a. Advantageously the cross section area of the inlet respectively outlet channels 114a, 114b, 116a, 116b substantially corresponds to the cross section area of the lumen 118. If the area of the at least one inlet and/or the at least one outlet channel is too small this increases the back-pressure which may be disadvantageous. It may especially be disadvantageous if the area of the at least one outlet channel 116 is too small since the increased back pressure increases the risk for ultra filtering through the membrane. Therefore it may be advantageous to provide several outlet channels, for example four outlet channels 116a-d as shown in FIGS. 3b, 3d. The inlet and/or outlet channels 114a, 114b, 116a, 116b may e.g. have a circular or oval cross section. In FIGS. 4b and 4d oval outlet channels 116a, 116b are shown. Several inlet respectively outlet channels may be placed after one another to increase the total area of the respective channel. To use an oval shape in comparison with a circular shape for the inlet and/or outlet channel has the advantage of enabling a larger cross section area of the channel while not increasing the amount of material cut away from the tube 102 in the transversal direction of the tube 102. The oval shape is preferably carried out as a circle that is cut in two halves along a centreline and where the two halves are placed at a distance from each other and the end points of the halves are connected with straight lines. See FIGS. 1-4 where different shapes and configurations of the inlet and outlet channels are illustrated. The distance from the edge of the membrane 104 to the edge of the first (in case of more than one inlet channel) inlet channel 114 is advantageously as short as possible. However, to ensure that no adhesive material 106 enters the inlet channel 114 the mentioned distance is advantageously about 1 mm. The above mentioned is also valid for the distance between the edge of the last (in case of more than one outlet channel) outlet channel and the edge of the membrane 104. The terms last and first referring to the flow direction of the perfusion fluid 126.

When the linear device 100 is used perfusion fluid 126 is pumped into the inlet end 122. The length of the outlet tube 102b (the length of the tube extending after, in the flow direction, the at least one outlet channel 116a, 116b) then must be adapted to the MWCO of the membrane to avoid ultra filtering. The higher the MWCO, the shorter the outlet tube 102b needs to be. Ultra filtering is a condition when the perfusion fluid 126 penetrates through the membrane 104. This may happen when the back pressure is too high in relation to the MWCO of the membrane that is used. To reduce the back pressure the length of the outlet tube 102b may be reduced, and/or as mentioned before, increase the number and/or cross sectional area of the at least one outlet channel 116.

At the outlet end 124 the dialysate 128 may be collected in an dialysate container 130 which is then put in an analysing instrument (not shown) for analysing the composition of the dialysate 128.

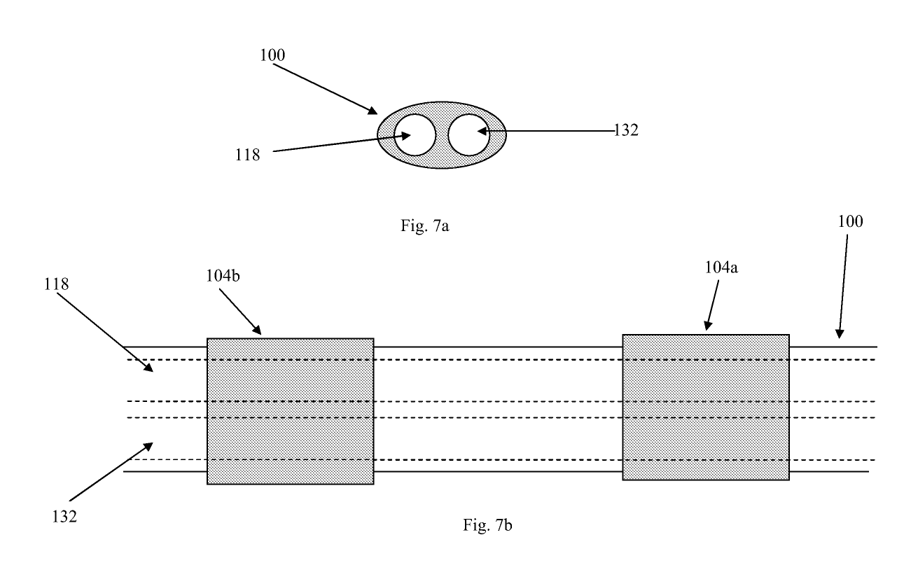

It is also possible with a linear device 100 having more than one membrane 104, e.g. two membranes 104a and 104b located after another, see FIGS. 7a, 7b. This may be an advantage when studying the effect of a drug administered to the body through one membrane 104a and where the effect of the drug may be studied through the other membrane 104b. In this case the linear device 100 comprises two lumen 118 and 132 where each lumen is connected to a respective microdialysis chamber 120a and 120b (not shown) and the lumens are separated from each other. The lumen 118 and 132 may in this case be placed next to each other so that the linear device gets a substantially oval or elliptical cross section or shape. In this way a multi lumen linear device does not need to be thicker or higher than a linear device having one lumen and the intervals for the lower height of a multi lumen linear device may be the same as previously mentioned for a single lumen linear device. Moreover, when applicable, what has been stated for the linear device having one lumen also is valid for a multi lumen linear device.

It may be advantageous to provide the linear device 100 with a detection element 134, e.g. a detection element possible to detect with ultrasound or computed tomography (CT). The element 134 may for example be a part of the blocking 110 and may comprise a wire of gold or acid-proof stainless steel for the detection with ultra sound. To provide the linear device 100 with a detection element may be advantageous since it enables the position of the linear device 100 to be detected and adjusted. If the linear device 100 comprises two blockings 110a, 111a and 110b, 111b the detection element 134 may be placed between these two blockings Reference sign 138 denotes a connection element for the linear device 100. The connection element 138 is used to connect the linear device 100 to a source of perfusion liquid, e.g. a pump (not shown).

A linear device 100 for microdialysis as described herein is adapted to enable a flow of a perfusion liquid 126 from the inlet end 122 of the tube 102, through an inlet lumen 118a, further through the at least one inlet channel 114a, 114b, then through the dialysis chamber 120. After the dialysis chamber 120 the perfusion liquid 126 (often called dialysate after the dialysis chamber 120) is enabled to flow through the at least one outlet channel 116a, 116b and then through the outlet lumen 118b connected to said chamber 120 towards an outlet end 124 of the tube 102.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

### REFERENCE SIGNS

Linear device—100
Tube—102
Semi-permeable membrane—104
Attachment means—106
Shrinking tube—108
Plug—110
Plug through hole—112
Inlet channel—114
Outlet channel—116
Lumen in tube 102-118
Dialysis chamber—120
Inlet end—122
Outlet end—124
Perfusion fluid—126
Dialysate—128
Dialysate container—130
Second lumen—132
Detection element—134
Layer of adhesive between tube 102 and membrane 104-136
Connection element for linear device 100-138

1. A linear device for microdialysis, comprising at least one dialysis chamber, a length of tubing having at least one lumen, at least one first element and at least one second element connecting one of said at least one lumen with one of said at least one dialysis chamber, wherein at least one tubular, sleeve-formed membrane is coaxially arranged outside the tubing periphery and extending at least partially along the periphery of the tubing, intermediate the ends of the tubing, and wherein the tubing extends at least through the membrane.

2. A linear device for microdialysis according to claim 1, wherein the tubing imparts sufficient rigidity to the linear device in order to eliminate the need for using supporting wires to support or protect the membrane when locating the linear device in target tissue.

3. A linear device for microdialysis according to claim 1, wherein the membrane extends sufficiently long along the periphery of the tubing to establish a suitable interaction between a perfusion fluid transported in the lumen and a surrounding body fluid, so that sufficient material can be collected from a target tissue and transported with the perfusion fluid for subsequent analysis.

4. A linear device for microdialysis according to claim 1, wherein the membrane is arranged on the tubing to provide said dialysis chamber between the outer surface of the length of tubing and the inner surface of the membrane.

5. A linear device for microdialysis according to claim 1 wherein said at least one first element comprises at least one inlet channel and said at least one second element comprises at least one outlet channel connecting one of said at least one lumen with one of said at least one dialysis chamber.

6. A linear device for microdialysis according to claim 5, wherein there is provided at least one blocking in the tubing between the at least one first element or inlet channel and the at least one second element or outlet channel, said at least one blocking the lumen for passage of liquid.

7. A method of manufacturing a linear device for microdialysis, the method comprising the following steps:
a. providing a length of tubing having at least one lumen,
b. providing at least one first element or inlet channel extending from the outside surface of the length of tubing to the at least one lumen,
c. providing at least one second element or outlet channel at a distance from the at least one first element or inlet channel, the at least one second element or outlet channel extending from the outside surface of the length of tubing to the at least one lumen,
d. providing at least one blocking between said at least one first element or inlet channel and the at least one second element or outlet channel, said blocking stopping the passage of liquid in the lumen,
e. arranging a tubular, sleeve-formed membrane coaxially outside the tubing periphery and extending at least partially along the periphery of the tubing, said membrane covering the at least one first element or inlet channel and the at least one second element or outlet channel,
f. sealingly attaching the membrane to the tubing.

8. A method of manufacturing according to claim 7 wherein the membrane is sealingly attached to the tubing by means of attachment means.

9. A method for studying a tissue, for example for studying the local effect of a drug substance in a tissue and/or for studying the condition in a tissue, wherein the method comprises the following steps:
a. invasively locating a linear device according to claim 1 in a target tissue,
b. providing a perfusion fluid in a lumen of said linear device,
c. permitting interaction between the perfusion fluid and the tissue by a membrane of said linear device,
d. collecting perfusion fluid having interacted with the tissue for analysis.

10. A method according to claim 9, wherein the linear device is used to study skin tissue.

11. A method according to claim 9, wherein the linear device is used to administer substances to tissue where the effect of the substance is studied by means of the linear device.

12. A method according to claim 9, wherein a first linear device is used to administer a drug substance to the target tissue and a second linear device is used to study the effect of the drug substance.

13. A method according to claim 8, wherein the attachment means comprises an adhesive material.
